(19) United States
(12) Patent Application Publication
(10) Pub. No.: US 2010/0174083 A1
(43) Pub. Date: Jul. 8, 2010

(54) METHOD FOR THE PREPARATION OF MONTELUKAST

(76) Inventors: Ales Halama, Pardubice (CZ); Josef Jirman, Praha (CZ)

Correspondence Address: Pearl Cohen Zedek Latzer, LLP, 1500 Broadway, 12th Floor, New York, NY, 10036, US

(21) Appl. No.: 12/522,407

(22) PCT Filed: Jan. 8, 2008
(86) PCT No.: PCT/CZ2008/000002

§ 371 Date: Mar. 17, 2010

(30) Foreign Application Priority Data
Jan. 9, 2007 (CZ) PV 2007-20

(51) Int. Cl. C07D 215/18

(52) U.S. Cl. 546/174

(57) ABSTRACT

A method of preparation of Montelukast of formula (I) by reaction of the compound of formula (III) and a compound of formula (IX), characterized in that the reaction is carried out in the presence of a base, an inert solvent and a component increasing selectivity of the process, especially of a polyether of general formula (XIII), wherein R stands for hydrogen or an alkyl and the value of n varies from 1 to 40, polyethyleneglycol of general formula (XIV), wherein n=1 to 40 or a crown-ether of formulae (XV), (XVI) or (XVII).

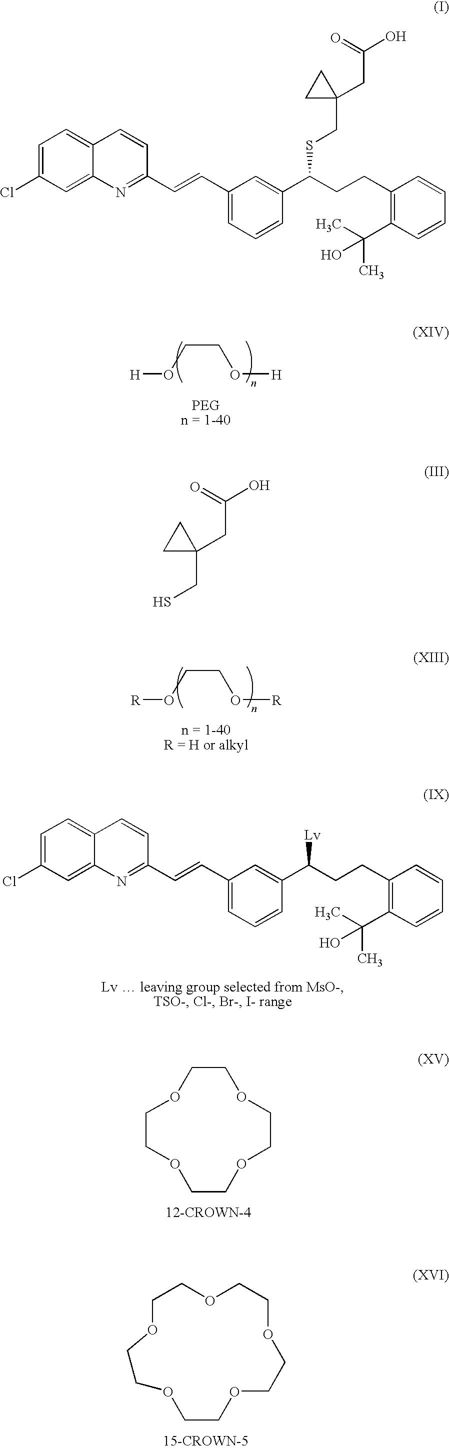

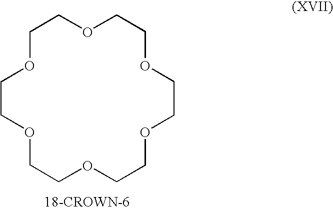

### TECHNICAL FIELD

The invention deals with a new preparation process of Montelukast of formula I, i.e. a substance that is used to prepare medicaments for the treatment of asthma and allergies.

### BACKGROUND ART

Montelukast, chemically [R-(E)]-1-[[[1-[3-[2-(7-chlor-2-quinolinyl)ethenyl]phenyl]-3-[2-(1-hydroxy-1-methylethyl)phenyl]propyl]thio]methyl]cyclopropaneacetic acid of formula I, is a well-known antiasthmatic and antiallergic drug. It is mainly the sodium salt of Montelukast described with formula II that is used in therapy.

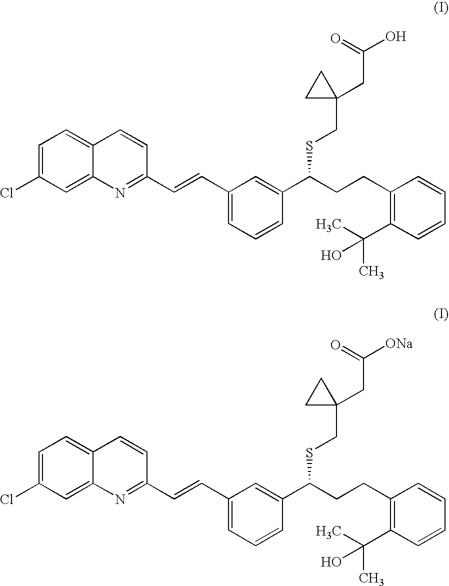

The sodium salt of Montelukast, its preparation and various forms, amorphous or crystalline, are described in a number of patents or applications, e.g. amorphous Montelukast Sodium is described in EP 0737186 B1 (MERCK, 1995), WO 03/066598 A1 (REDDY), WO 2004/108679 A1 (MOREPHEN, 2004), WO 2005/074893 A1 (CHEMAGIS). Crystalline polymorphs of Montelukast Sodium are dealt with by WO 2004/091618 A1 (MERCK), WO 2005/075427 A2 (TEVA).

The first method of chemical synthesis of Montelukast (I) was described in the patent no. EP 0480717 B1 (MERCK, 1992) and subsequently in specialized literature (M. Labele, Bioorg. Med. Chem. Lett. 5 (3), 283-288 (1995)). From the point of view of chemical synthesis of Montelukast of formula I the key step is binding of two building blocks (intermediates) with the use of a newly created bond between the carbon and sulphur atoms while the absolute configuration is maintained or completely inverted.

In principle, there are two basic methods of carrying out the key step of synthesis of Montelukast (I), which are indicated in Scheme 1. The first method represents formation of the C—S bond indicated as method A) in the diagram, the second one as method B). Both the basic methods can be further extended with a number of variants that are mainly based on alternating the order of reaction stages. The solution based on method A) is described in the following patents: EP 0480717 B1 (MERCK, 1992), EP 0737186 B1 (MERCK, 1995), US 2005/0234241 A1 (REDDY, 2005), WO 2005/105751 A1 (TEVA, 2005), US 2005/0107612 A1 (REDDY, 2005). The solution based on method B) is described in two patent applications of the SYNTHON Company: WO 2005/105749 A2 (SYNTHON, 2005), WO 2005/105750 A1 (SYNTHON, 2005).

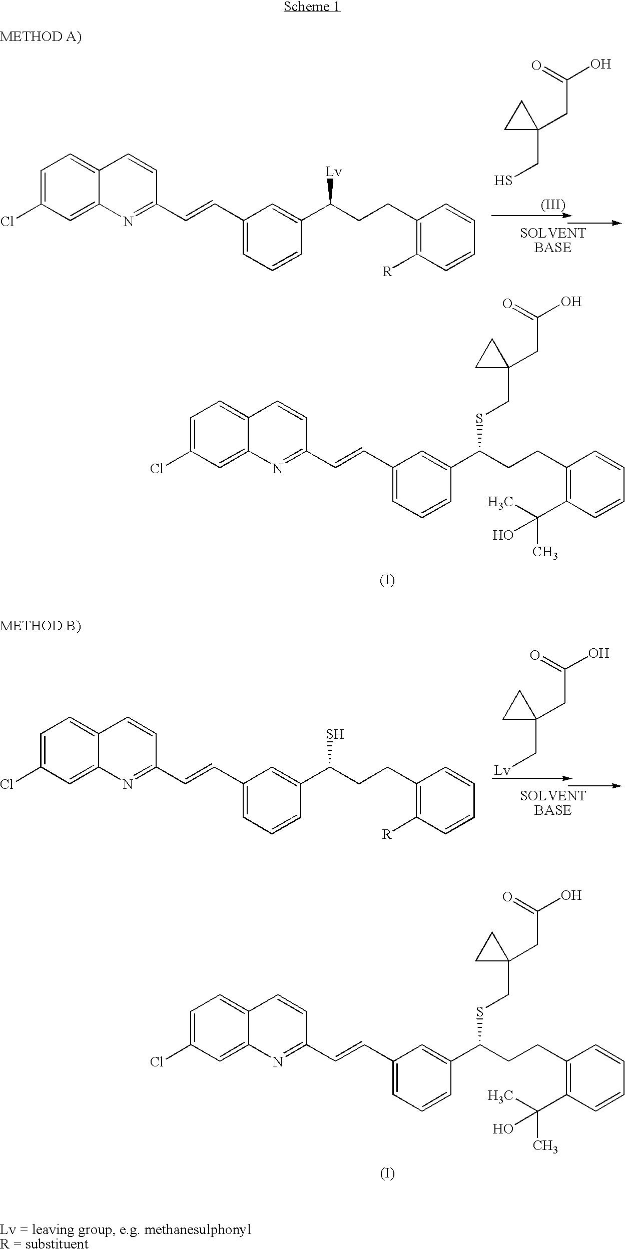

In practice mainly the solution is used that is characterized by the use of the dilithium salt of [1-(mercaptomethyl)cyclopropyl]acetic acid of formula III-diLi. The process is described in patent No. EP 0737186 B1 (MERCK, 1995); in an abbreviated form it is described in Scheme 2. This solution also comprises a method of purification of crude Montelukast of formula I via its salt with dicyclohexylamine and also a method of obtaining the sodium salt of Montelukast of formula II.

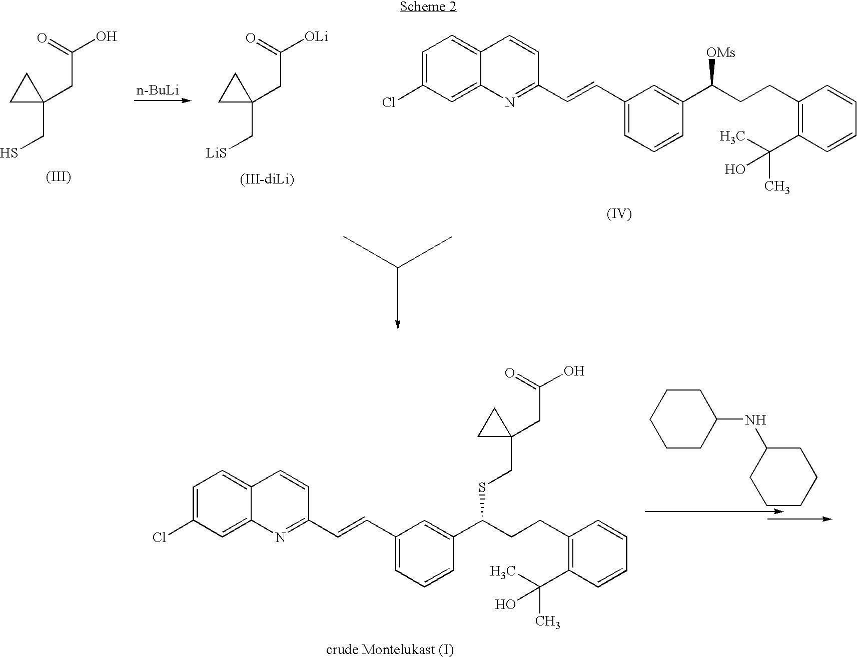

The process of preparation of Montelukast of formula I as well as the quality of the obtained product is affected by undesired reactions that both the starting materials and the product itself may be subject to depending on the process conditions. Literature describes increased sensitivity of the target substance to oxygen (see equation (1)) the substance of chemical formula (V) being described as the main product of oxidation of Montelukast of formula I (E. D. Nelson, J. Pharm. Sci. 95, 1527-1539 (2006), C. Dufresne, J. Org. Chem. 1996, 61, 8518-8525)). Contamination of the product with this or other impurities is most undesirable. For this reason processes of preparation of the target substance with the exclusion of oxygen are used, i.e. under the protective atmosphere of an inert gas (e.g. nitrogen in accordance with EP 0737186 B1).

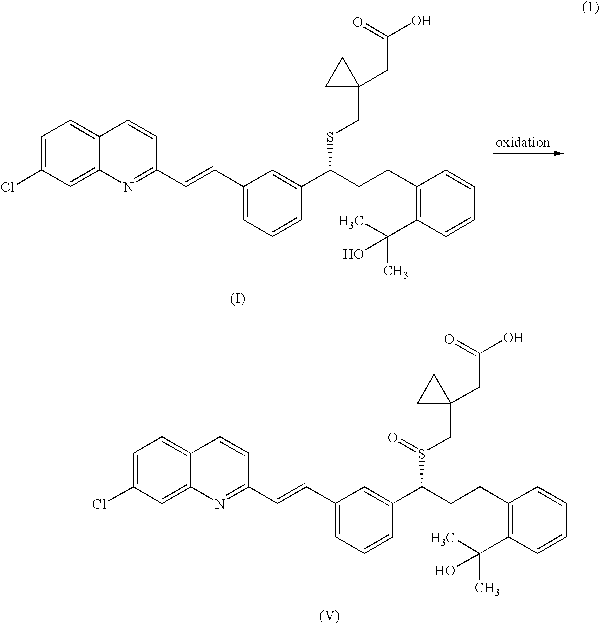

Another source of chemical contamination of Montelukast is instability of the normally used starting material described with formula IV. This substance is subject mainly to three unwanted reactions—hydrolysis, elimination and cyclization with the formation of impurities described with formulas (VI-VIII), see Scheme 3 (J. O. Egekeze, Anal. Chem. 1995, 67, 2292-2295).

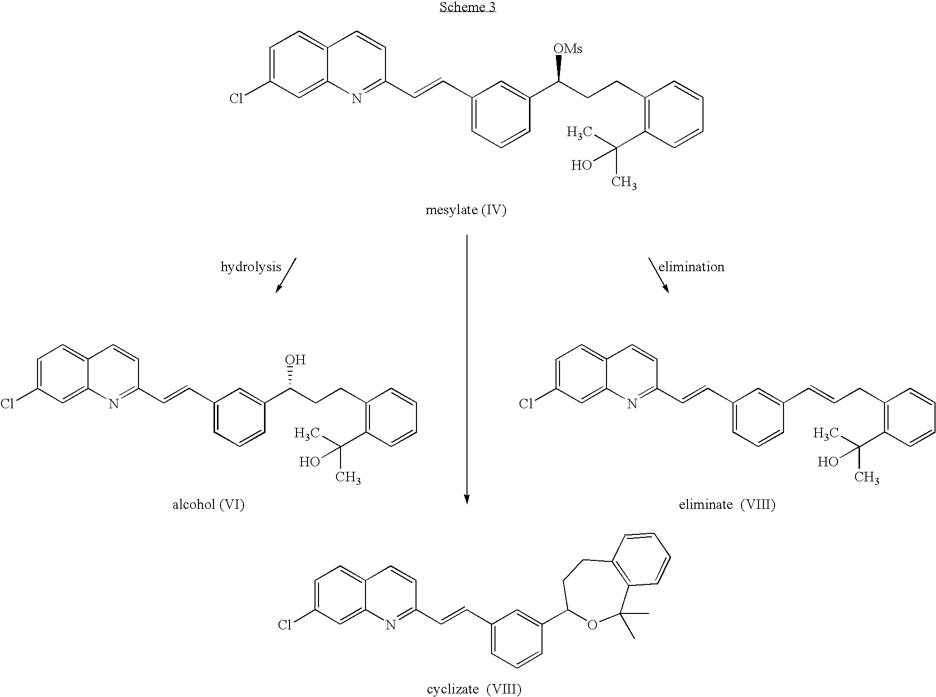

Hydrolysis can be prevented with the use of anhydrous organic solvents (e.g. THF in accordance with EP 0737186 B1), elimination can be prevented by performing the process at lower temperatures (below −5° C. in accordance with EP 0737186 B1), cyclization is prevented with the use of protective groups (e.g. tetrahydropyranyl (in short THP), M. Labele, Bioorg. Med. Chem. Lett. 5 (3), 283-288 (1995)), which however, increases the number of synthetic steps (introduction of the protective group and subsequently its deprotection), see Scheme 4.

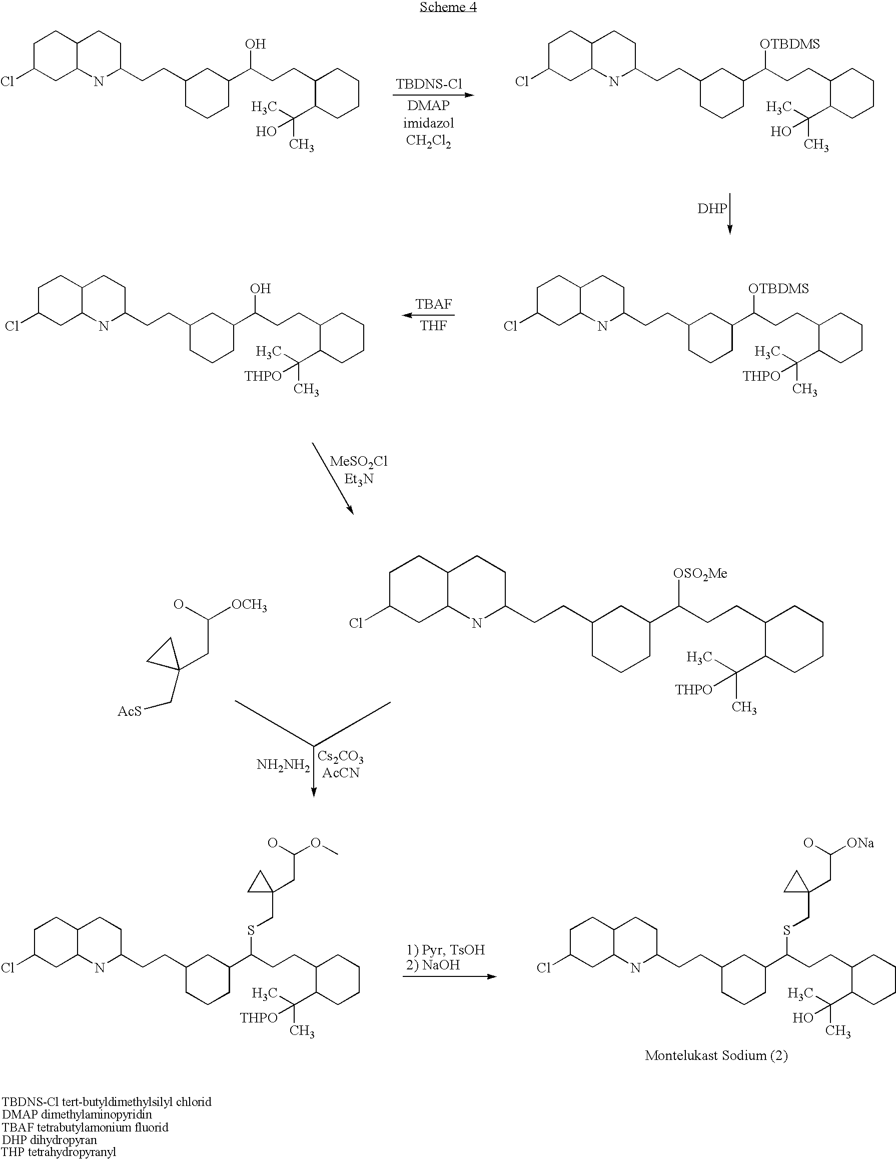

The present solution represents a new and advantageous method of carrying out the key substitution reaction that leads to Montelukast of formula I. The present procedure is distinguished from the previous solutions in the use of linear or cyclic polyethers, which ensure higher selectivity of the process and reduce the formation of undesired side products.

### SUMMARY OF THE INVENTION

The invention relates to a new method of carrying out the substitution reaction that represents the key stage in the process of preparation of Montelukast of formula I. This reaction is described by the following equation (2).

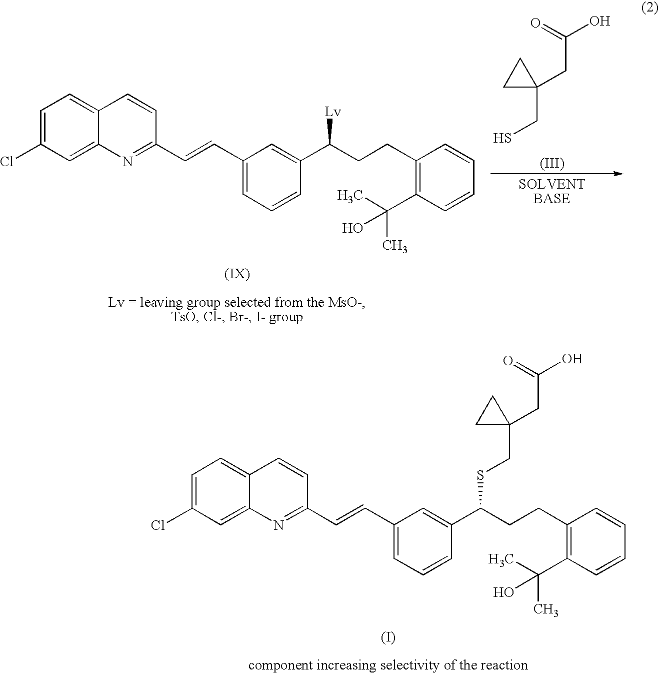

As the starting material of the present process of preparation of Montelukast of formula I a substance described with general formula IX can be used, preferably the substance of formula IV containing the methanesulphonic group as the leaving group. The starting material of formula IV is prepared e.g. in the way described in patents no. EP 0737186 B1 (MERCK, 1995), WO 2005/105751 A1 (TEVA, 2005) in accordance with the equation (3).

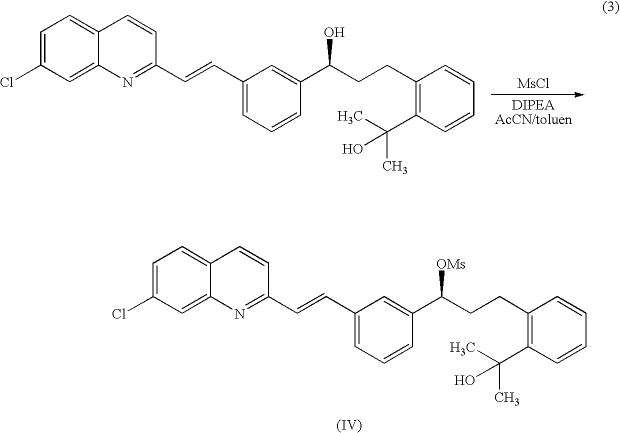

Another starting material of the present process is [1-(mercaptomethyl)cyclopropyl]acetic acid of formula III, which is converted to the corresponding salt by the effect of two base equivalents directly in the reaction mixture. This conversion is described by the equation (4). This salt only represents the active form of the reagent. Depending on the used ion the characteristics of these salts differ, mainly their solubility in the reaction environment. While the dilithium salt (III-diLi) is soluble for the most part in the used solvents, the disodium (III-diNa) and dipotassium (III-diK) salts do not dissolve for the greater part, which reduces the current concentration of the active agent in the solution. The resulting effect is deceleration of the required substitution reaction and reduction of its selectivity.

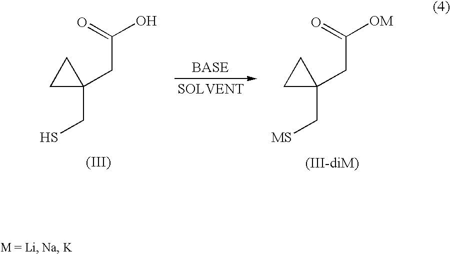

The process of the invention is further characterized with the use of bases that convert the acid of formula III to the respective salts (III-diM, where M indicates an alkali metal). As the base various substances can be used, e.g. organometallic compounds as well as alkali metal hydroxides and alkoxides of formula X. To ensure acceptable solubility suitable bases are alkoxides of metals, especially alkoxides characterized by a branched alkyl chain in their structure, e.g. tert-butoxides and tert-amylates of alkali metals (Li, Na, K) described with formulas XI and XII.

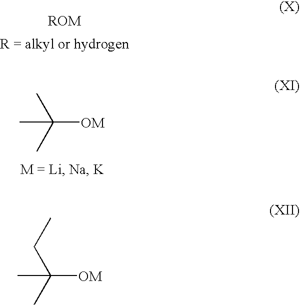

As the reaction environment inert organic solvents can be used, especially aromatic hydrocarbons, ethers, esters, amides, or sulphoxides or their mixtures in any proportions. E.g. the following solvents are suitable: toluene, benzene, tetrahydrofuran, methyltetrahydrofuran, dimethylcarbonate, dimethylformamide or dimethylsulphoxide. Using a mixture of toluene and tetrahydrofuran is particularly advantageous.

Unlike the previous solutions the present process makes use of advantageous properties of the polyethers of general formula XIII that can be either linear of formula XIV or cyclic of formulas XV-XVII. These substances play the role of phase transfer catalysts of complexing agents that solvate metal ions, increasing the solubility and reactivity of the used nucleophilic reagent, i.e. (III-diM). The increased reactivity of the reagent results in higher selectivity of the process, i.e. the impact of unwanted competitive reactions that lead to formation of impurities is suppressed. Thanks to the addition of the polyether to the reaction mixture the key synthetic stage occurs in a solution, not in the suspension phase.

As suitable linear polyethers polyethyleneglycols of formula XIV referred to with the PEG abbreviation can be used, mutually differing in their chain lengths or, more precisely, mixtures of polyethyleneglycols specified by the average molar weight, e.g. PEG-600 or PEG-1500. CROWN ethers of formulas XV-XVII with different cycle sizes can serve as suitable cyclic polyethers.

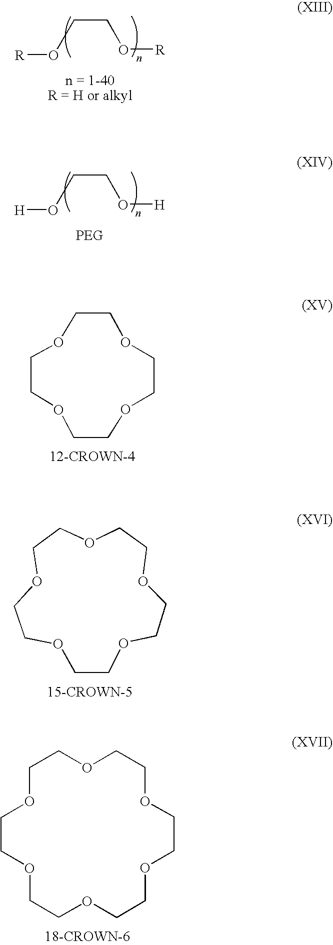

The reactions leading to the target substance of formula I were performed in accordance with the procedure of the present invention in such a way that first the carboxylic acid of formula III was mixed with the base of general formula X and polyether of formula XIII in the environment of an inert organic solvent and under an inert gas atmosphere. The obtained mixture was cooled down below −5° C. and then a solution of the substance of formula IV in a suitable organic solvent was added dropwise. Further, the reaction mixture was stirred under an inert atmosphere at the temperature of −5 to 40° C. for several hours and samples were drawn continuously to determine the conversion and selectivity of the substitution reaction. The crude product of formula I was finally isolated from the reaction environment with the yields of 65-75% by commonly used processes that have been described before in patents nos. EP 0737186 B1, US 2005/0234241 A1, WO 2005/105751 A1.

The process of the present invention is advantageous especially in that due to the addition of the polyether of formula XIII to the reaction mixture the desirable reaction (substitution) is accelerated as compared to undesired conversions (hydrolysis, elimination, cyclization). This way the negative influence of competitive reactions on the final composition of the reaction mixture at the end of the reaction, i.e. at the time of consumption of the starting substance, e.g. the substance of formula IV, is limited. Cyclic or linear polyethers of general formula XIII increase both solubility and reactivity (nucleophility) of the reagent (III-diM), which subsequently increases selectivity of the desired reaction. In carrying the key stage of preparation of Montelukast of formula I out in accordance with the present invention, the resulting composition of the reaction mixture is conveniently controlled without the necessity of long-term cooling or use of protective groups, which is a solution associated with an increase of the number of reaction stages. Besides the limitation of the content of impurities in the crude product our solution is also characterized by better utilization of the starting substances, i.e. the very expensive substance of formula IV in particular is not excessively consumed by undesired reactions. The advantageousness of our solution is demonstrated in the examples and also in FIGS. 1 and 2 in the appendixes and in table 1. FIG. 1 compares the composition of reaction mixtures of a reaction without the use of a cyclic or linear polyether and a reaction when these substances are added to the reaction mixture. FIG. 2 compares the dependence of the contents of Montelukast in reaction mixtures on different conditions of the substitution reactions (in accordance with examples 3, 4 and 6). Table 1 compares conversions and selectivity measured after 24 hours from the mixing of the components in various modifications of the substitution reaction leading to Montelukast of formula I in accordance with examples 1-9.

### BRIEF DESCRIPTION OF DRAWINGS

FIG. 1 shows HPLC chromatograms of reaction mixtures of the substitution reaction of mesylate (IV)
A in accordance with example 4 after 48 hours (with the component increasing selectivity of the reaction: 18-CROWN-6).
B in accordance with example 3 after 48 hours (without addition of the component increasing selectivity of the reactions).
   Sequence of peaks: 1. alcohol (VI), 2. mesylate (IV), 3. Montelukast (1), 4. eliminate (VII), 5. unknown impurity, 6. cyclizate (VIII).

FIG. 2 shows the dependence of contents of Montelukast in the reaction mixture in substitution reactions carried out under various conditions (in accordance with examples 3, 4 and 6).

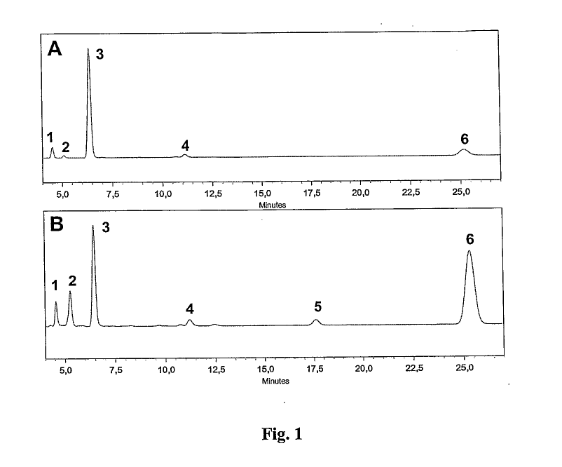

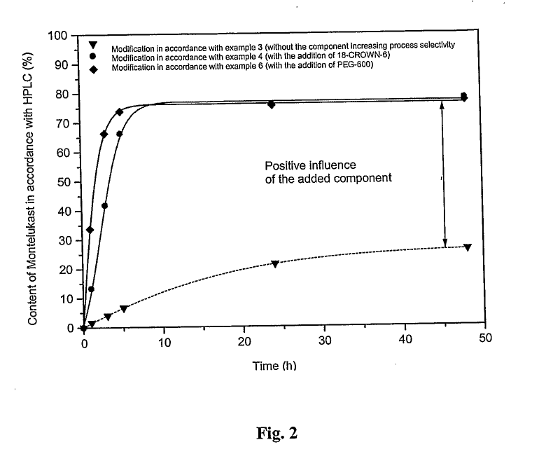

### EXAMPLES

The subject matter of the invention will be explained in a more detailed way with the use of the following examples, which however, do not have any influence on the scope of the invention defined in the claims.

### Example 1

In 20 ml of toluene[1-(mercaptomethyl)cyclopropyl]acetic acid (0.28 g) and the base (lithium tert-butoxide, 0.31 g) were mixed and the mixture wan stirred in argon atmosphere and cooled to ca. −10° C. A solution of 2-(3-(S)-(3-(2-(7-chloroquinolinyl)-ethenyl)phenyl)-3-methanesulphonyloxypropyl)phenyl-2-propanol (1 g) in 5 ml of tetrahydrofuran was subsequently added to the obtained slurry. The reaction mixture was stirred gradually from −10° C. up to the laboratory temperature for an hour. Then it was stirred at the laboratory temperature (about 21° C.) for several hours. The reaction mixture was continuously analyzed with HPLC.

### Example 2

In 20 ml of toluene[1-(mercaptomethyl)cyclopropyl]acetic acid (0.28 g), the base (lithium tert-butoxide, 0.31 g) and 12-crown-4 (0.33 g) were mixed and the mixture wan stirred in argon atmosphere and cooled to ca. −10° C. A solution of 2-(3-(S)-(3-(2-(7-chloroquinolinyl)-ethenyl)phenyl)-3-methanesulphonyloxypropyl)phenyl-2-propanol (1 g) in 5 ml of tetrahydrofuran was subsequently added to the obtained slurry. The reaction mixture was stirred gradually from −10° C. up to the laboratory temperature for an hour. Then it was stirred at the laboratory temperature (about 21° C.) for several hours. The reaction mixture was continuously analyzed with HPLC.

### Example 3

In 20 ml of toluene[1-(mercaptomethyl)cyclopropyl]acetic acid (0.28 g) and the base (sodium tert-butoxide, 0.36 g) were mixed and the mixture wan stirred in argon atmosphere and cooled to ca. −10° C. A solution of 2-(3-(S)-(3-(2-(7-chloroquinolinyl)-ethenyl)phenyl)-3-methanesulphonyloxypropyl)phenyl-2-propanol (1 g) in 5 ml of tetrahydrofuran was subsequently added to the obtained slurry. The reaction mixture was stirred gradually from −10° C. up to the laboratory temperature for an hour. Then it was stirred at the laboratory temperature (about 21° C.) for several hours. The reaction mixture was continuously analyzed with HPLC.

### Example 4

In 20 ml of toluene[1-(mercaptomethyl)cyclopropyl]acetic acid (0.28 g), the base (sodium tert-butoxide, 0.36 g) and 18-crown-6 (0.50 g) were mixed and the mixture wan stirred in argon atmosphere and cooled to ca. −10° C. A solution of 2-(3-(S)-(3-(2-(7-chloroquinolinyl)-ethenyl)phenyl)-3-methanesulphonyloxypropyl)phenyl-2-propanol (1 g) in 5 ml of tetrahydrofuran was subsequently added to the obtained slurry. The reaction mixture was stirred gradually from −10° C. up to the laboratory temperature for an hour. Then it was stirred at the laboratory temperature (about 21° C.) for several hours. The reaction mixture was continuously analyzed with HPLC.

### Example 5

In 20 ml of toluene[1-(mercaptomethyl)cyclopropyl]acetic acid (0.28 g), the base (sodium tert-butoxide, 0.36 g) and 15-crown-5 (0.41 g) were mixed and the mixture wan stirred in argon atmosphere and cooled to ca. −10° C. A solution of 2-(3-(S)-(3-(2-(7-chloroquinolinyl)-ethenyl)phenyl)-3-methanesulphonyloxypropyl)phenyl-2-propanol (1 g) in 5 ml of tetrahydrofuran was subsequently added to the obtained slurry. The reaction mixture was stirred gradually from −10° C. up to the laboratory temperature for an hour. Then it was stirred at the laboratory temperature (about 21° C.) for several hours. The reaction mixture was continuously analyzed with HPLC.

### Example 6

In 20 ml of toluene[1-(mercaptomethyl)cyclopropyl]acetic acid (0.28 g), the base (sodium tert-butoxide, 0.36 g) and PEG-600 (1.1 g) were mixed and the mixture wan stirred in argon atmosphere and cooled to ca. −10° C. A solution of 2-(3-(S)-(3-(2-(7-chloroquinolinyl)-ethenyl)phenyl)-3-methanesulphonyloxypropyl)phenyl-2-propanol (1 g) in 5 ml of tetrahydrofuran was subsequently added to the obtained slurry. The reaction mixture was stirred gradually from −10° C. up to the laboratory temperature for an hour. Then it was stirred at the laboratory temperature (about 21° C.) for several hours. The reaction mixture was continuously analyzed with HPLC.

### Example 7

In 20 ml of toluene[1-(mercaptomethyl)cyclopropyl]acetic acid (0.28 g), the base (sodium tert-butoxide, 0.36 g) and PEG-1500 (2.7 g) were mixed and the mixture wan stirred in argon atmosphere and cooled to ca. −10° C. A solution of 2-(3-(S)-(3-(2-(7-chloroquinolinyl)-ethenyl)phenyl)-3-methanesulphonyloxypropyl)phenyl-2-propanol (1 g) in 5 ml of tetrahydrofuran was subsequently added to the obtained slurry. The reaction mixture was stirred gradually from −10° C. up to the laboratory temperature for an hour. Then it was stirred at the laboratory temperature (about 21° C.) for several hours. The reaction mixture was continuously analyzed with HPLC.

### Example 8

In 20 ml of toluene[1-(mercaptomethyl)cyclopropyl]acetic acid (0.28 g) and the base (potassium tert-amylate, 0.42 g) were mixed and the mixture wan stirred in argon atmosphere and cooled to ca. −10° C. A solution of 2-(3-(S)-(3-(2-(7-chloroquinolinyl)-ethenyl)phenyl)-3-methanesulphonyloxypropyl)phenyl-2-propanol (1 g) in 5 ml of tetrahydrofuran was subsequently added to the obtained slurry. The reaction mixture was stirred gradually from −10° C. up to the laboratory temperature for an hour. Then it was stirred at the laboratory temperature (about 21° C.) for several hours. The reaction mixture was continuously analyzed with HPLC.

### Example 9

In 20 ml of toluene[1-(mercaptomethyl)cyclopropyl]acetic acid (0.28 g), the base (potassium tert-amylate, 0.42 g) and 18-crown-6 (0.50 g) were mixed and the mixture wan stirred in argon atmosphere and cooled to ca. −10° C. A solution of 2-(3-(S)-(3-(2-(7-chloroquinolinyl)-ethenyl)phenyl)-3-methanesulphonyloxypropyl)phenyl-2-propanol (1 g) in 5 ml of tetrahydrofuran was subsequently added to the obtained slurry. The reaction mixture was stirred gradually from −10° C. up to the laboratory temperature for an hour. Then it was stirred at the laboratory temperature (about 21° C.) for several hours. The reaction mixture was continuously analyzed with HPLC.

TABLE 1
Comparing conversions and selectivities after 24 hours from
mixing of the components in different modifications of the
substitution reaction leading to Montelukast (I)

| Sample | Component increasing | Conversion | |
|---|---|---|---|
| no. | reaction selectivity | (%) | Selectivity (%) |
| 1 | — | 97.9 | 60.8 |
| 2 | 12-CROWN-4 | 97.3 | 59.6 |
| 3 | — | 82.3 | 26.0 |
| 4 | 18-CROWN-6 | 96.1 | 79.0 |
| 5 | 15-CROWN-5 | 94.6 | 77.5 |
| 6 | PEG-600 | 93.2 | 81.1 |
| 7 | PEG-1500 | 81.4 | 68.2 |
| 8 | — | 95.0 | 9.6 |
| 9 | 18-CROWN-6 | 92.7 | 51.5 |

### Description of the Analytical Method

Conversions and selectivities in the present process of preparation of Montelukast were determined with the use of the HLPC method. The chromatograms were measured with the EliteLachrom device made by the Hitachi Company. As the mobile phase a mixture of acetonitrile (80%) and of a 0.1M aqueous solution of ammonium formate adjusted to pH 3.6 with formic acid (20%). The measurements were performed in an isocratic mode with the flow rate of the mobile phase of 1.5 ml/min.

1. A method for the preparation of Montelukast according to formula I by reacting the compound of formula III with a compound of formula IX wherein the reaction is carried out in the presence of a base, an inert solvent and a component that increases selectivity of the process.

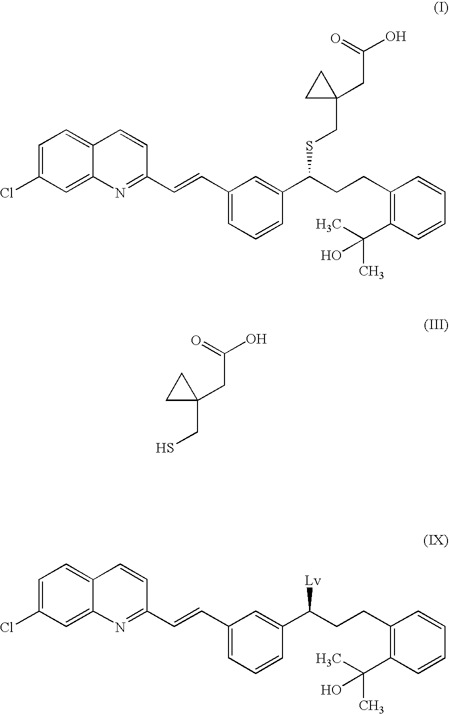

2. The method according to claim 1, wherein the component that increases process selectivity is a polyether of general formula XIII is used, wherein R stands for hydrogen or an alkyl and the value of n varies from 1 to 40.

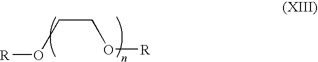

3. The method according to claim 2, wherein the component that increases process selectivity is a polyethyleneglycol of general formula XIV, wherein the value of n varies from 1 to 40.

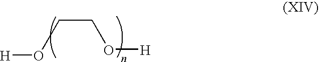

4. The method according to claim 2, wherein the component that increases process selectivity is a CROWN ether according to any one of the structures set forth in formula (XV), (XVI) or (XVII).

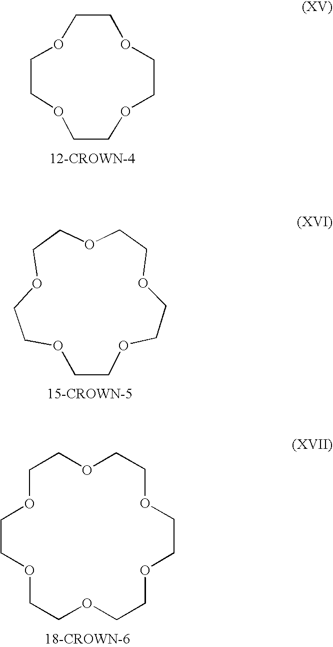

5. A method for the preparation of Montelukast according to formula I by reacting the compound of formula III with a compound of formula IV, wherein Ms stands for the methanesulphonyl leaving group, and wherein the reaction is carried out in the presence of a base, an inert solvent and a component that increases selectivity of the process.

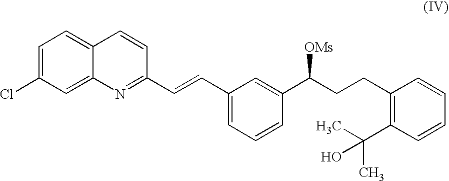

6. The method according to claim 1, wherein the base is an alkali metal alkoxide according to the structure set forth in general formula ROM, wherein R stands for an alkyl and M stands for an alkali metal selected from the group consisting of Li, Na and K.

7. The method according to claim 6, wherein the base is an alkali metal alkoxide according to the structure set forth in formula (XI) or (XII), wherein M stands for an alkali metal selected from the group consisting of Li, Na and K.

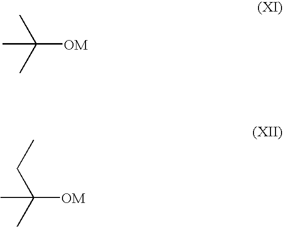

8. The method according to claim 1, wherein the inert organic solvent is an aromatic hydrocarbon, ether, ester, amide or sulphoxide, or a mixture thereof.

9. The method according to claim 8, wherein the inert organic solvent is toluene, benzene, tetrahydrofuran, 2-methyltetrahydrofuran, dimethylcarbonate, dimethylformamide or dimethylsulphoxide or a mixture thereof.

10. The method according to claim 9, wherein the inert organic solvent is a mixture of toluene and tetrahydrofuran.

11. The method according to claim 5, wherein the carboxylic acid of formula (III) is mixed with a base of the general formula (X) and a polyether of formula (XIII) in the environment of an inert organic solvent and under an inert gas atmosphere; the obtained mixture is subsequently cooled and a solution of the compound of formula (IV) in an organic solvent is added dropwise; the reaction mixture being further stirred under the inert gas atmosphere until the consumption of the starting compounds.

12. The method according to claim 11, wherein the reacting components are mixed under cooling conditions, and the reaction is subsequently continued at the temperatures of the reaction mixture from −5 to +40° C.

13. The method according to claim 11, wherein the reacting components are mixed under at a temperature below −5° C., and the reaction is subsequently continued at the temperatures of the reaction mixture from about 20 to 25° C.
